(19) United States
(12) Patent Application Publication
(10) Pub. No.: US 2026/0108671 A1
(43) Pub. Date: Apr. 23, 2026

(54) METHODS OF RETRIEVING CONTRAST MEDIA

(71) Applicant: Boston Scientific Scimed, Inc., Maple Grove, MN (US)

(72) Inventors: David Ryan, Claremorris (IE); John Lardner, Galway (IE); Gavin O'Brien, Dublin (IE); Tim O'Connor, Galway (IE)

(73) Assignee: BOSTON SCIENTIFIC SCIMED, INC., Maple Grove, MN (US)

(21) Appl. No.: 19/361,320

(22) Filed: Oct. 17, 2025

Related U.S. Application Data
(60) Provisional application No. 63/708,508, filed on Oct. 17, 2024.

(51) Int. Cl. A61M 5/00; A61K 49/04; A61K 49/18; A61M 1/00; A61M 25/00

(52) U.S. Cl. CPC A61M 5/007; A61K 49/0438; A61K 49/1818; A61M 1/85; A61M 25/0043; A61M 2025/0057; A61M 2205/0272; A61M 2205/75

(57) ABSTRACT

A location within a patient may be visualized while reducing potential impact on the patient's anatomy. A contrast media may be injected into a location within the patient's vasculature. The location may be viewed fluoroscopically. At least some of the injected contrast media may be recaptured in order to reduce a quantity of contrast media reaching the patient's anatomy.

### CROSS REFERENCE TO RELATED APPLICATIONS

This application claims the benefit of priority of U.S. Provisional Application No. 63/708,508 filed Oct. 17, 2024, the entire disclosure of which is hereby incorporated by reference.

### TECHNICAL FIELD

The present disclosure relates generally to methods and apparatuses for various medical devices. More particularly, the disclosure relates to different methods of use and recapture of a contrast media.

### BACKGROUND

Contrast media is used for fluoroscopic visibility during a variety of different medical procedures. Implantation of an artificial cardiac valve is an example of a medical procedure that can utilize contrast media. As an example, contrast media may be used to visualize form and function of a native cardiac valve. Contrast media may also be used to visualize valve function of an implanted cardiac valve. The volume of contrast media injected may vary from procedure to procedure, and from patient to patient. Unfortunately, excessive contrast media may not be well-tolerated by some patients. Of the known methods of using contrast media, each has certain advantages and disadvantages. There is an ongoing need to provide alternate methods for use of contrast media that provide desired visibility while reducing the volume of contrast media that remains within the patient.

### SUMMARY

The disclosure is directed to several alternative designs, materials and methods of manufacturing medical device structures and assemblies, and the use thereof. An example may be found in a method for visualizing a location within a patient while reducing potential impact on the patient's anatomy. The method includes injecting a contrast media into a location within the patient's vasculature, viewing the location fluoroscopically, and recapturing at least some of the injected contrast media.

Alternatively or additionally, recapturing at least some of the injected contrast media may occur near the location.

Alternatively or additionally, recapturing at least some of the injected contrast media may occur downstream of the location.

Alternatively or additionally, the injecting step may temporally overlap with the recapturing step.

Alternatively or additionally, the viewing step may temporally overlap with the recapturing step.

Alternatively or additionally, the contrast media may be injected into the location using a first lumen fluidly coupled with one or more injection ports.

Alternatively or additionally, recapturing at least some of the injected contrast media near the location may include using a second lumen fluidly coupled with one or more aspiration ports.

Alternatively or additionally, the first lumen may extend through a first catheter and the second lumen may extend through a second catheter.

Alternatively or additionally, the first lumen and the second lumen may both extend through a single catheter, with the one or more injection ports disposed distally of the one or more aspiration ports.

Alternatively or additionally, recapturing at least some of the injected contrast media may include capturing the injected contrast media within a filter.

Alternatively or additionally, the filter may include a collapsible frame.

Alternatively or additionally, the contrast media may include a contrast agent bound to magnetic particles, and the filter may include an electromagnetic or a ferromagnetic surface.

Alternatively or additionally, the contrast agent may include iohexol.

Alternatively or additionally, the magnetic particles may include superparamagnetic iron oxide nanoparticles (SPIONs).

Another example may be found in an assembly for visualizing a location within a patient while reducing potential impact on the patient's anatomy. The assembly includes an injection catheter and an aspiration catheter. The injection catheter includes an injection lumen extending therethrough and one or more injection ports that are fluidly coupled with the injection lumen for injecting contrast media. The aspiration catheter includes an aspiration lumen extending therethrough and one or more aspiration ports that are fluidly coupled with the aspiration lumen.

Alternatively or additionally, the injection lumen may be adapted to be fluidly coupled with a source of contrast media and the aspiration lumen may be adapted to be fluidly coupled with a source of vacuum.

Another example may be found in a catheter. The catheter includes an elongate shaft having an injection lumen extending through the elongate shaft and an aspiration lumen extending through the elongate shaft. One or more injection ports are fluidly coupled with the injection lumen for injecting contrast media. One or more aspiration ports are fluidly coupled with the aspiration lumen, the one or more aspiration ports disposed proximally of the one or more injection ports.

Alternatively or additionally, the catheter may further include a collapsible filter that is secured to the elongate shaft near the one or more aspiration ports.

Alternatively or additionally, the catheter may further include a delivery lumen that is adapted to accommodate an implantable medical device advanced within the delivery lumen.

Alternatively or additionally, the catheter may further include a proximal hub. The proximal hub may include a first fitting that is fluidly coupled with the injection lumen and that may be adapted to be connected with a source of contrast media and a second fitting that is fluidly coupled with the aspiration lumen and that may be adapted to be connected with a source of vacuum.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative system for injecting and recapturing contrast media;

FIG. 1A is a cross-sectional view taken along line 1A-1A of FIG. 1;

FIG. 1B is a cross-sectional view taken along line 1B-1B of FIG. 1;

FIG. 2 is a schematic view of an illustrative magnetic filter in use;

FIG. 3 is a schematic view of an illustrative physical filter, before the contrast media has reached the physical filter;

FIG. 4 is a schematic view of the illustrative physical filter of FIG. 3, after the contrast media has reached the physical filter;

FIG. 5 is a schematic view of an illustrative multi-lumen catheter for injecting and recapturing contrast media, shown before vacuum is applied;

FIG. 5A is a cross-sectional view taken along line 5A-5A of FIG. 5.

FIG. 6 is a schematic view of the illustrative multi-lumen catheter of FIG. 5, shown after vacuum is applied; and

FIG. 7 is a schematic view of the illustrative multi-lumen catheter of FIG. 5, shown after the contrast media has been recaptured.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

Contrast media may be introduced during a variety of different medical procedures in order to increase visibility under fluoroscopy. In some cases, the contrast media helps to increase the visibility of anatomical structures in order to more accurately perform a medical procedure. In some cases, a patient may be sensitive to damage that may possibly occur if/when the contrast media reaches specific anatomy such as their kidneys. In some cases, it may be beneficial to limit how much contrast media reaches a patient's anatomy (i.e., kidneys) in order to reduce the chances of possible damage. In some instances, a method for visualizing a location within a patient while reducing potential impact on the patient's anatomy (i.e., kidneys) includes injecting a contrast media into a location within the patient and viewing the location fluoroscopically. At least some of the injected contrast media is recaptured during or after the procedure. In some cases, the targeted location within the patient may be a circulatory system (e.g., vasculature, heart, etc.).

In some cases, recapturing at least some of the injected contrast media may occur near the location where the media was injected. In some cases, recapturing at least some of the injected contrast media may occur downstream of the injected location. In some cases, the injecting step may temporally overlap with the recapturing step. In some cases, the viewing step may temporally overlap with the recapturing step. In some cases, recapturing at least some of the injected contrast media may include capturing the injected contrast media within a filter. In some cases, the filter may include a collapsible frame or support. In some cases, the contrast media may include a contrast agent that is bound to magnetic particles, and the filter may include an electromagnetic or a ferromagnetic surface. As an example, the contrast agent may include iohexol and the magnetic particles may include superparamagnetic iron oxide nanoparticles (SPIONs).

In some cases, the contrast media may be injected into the location using a first lumen fluidly coupled with one or more injection ports. Recapturing at least some of the injected contrast media near the location may include using a second lumen fluidly coupled with one or more aspiration ports. As an example, the first lumen may extend through a first catheter and the second lumen may extend through a second catheter. As another example, the first lumen and the second lumen may both extend through a single catheter, with the one or more injection ports disposed distally of the one or more aspiration ports.

In some instances, an assembly for visualizing a location within a patient while reducing potential impact on the patient's anatomy may include an injection catheter and an aspiration catheter. The injection catheter may include an injection lumen extending therethrough and one or more injection ports that are fluidly coupled with the injection lumen for injecting contrast media. The aspiration catheter may include an aspiration lumen extending therethrough and one or more aspiration ports that are fluidly coupled with the aspiration lumen. In some cases, the injection lumen may be adapted to be fluidly coupled with a source of contrast media and the aspiration lumen may be adapted to be fluidly coupled with a source of vacuum.

In some instances, a catheter includes an elongate shaft. An injection lumen extends through the elongate shaft. One or more injection ports are fluidly coupled with the injection lumen for injecting contrast media. An aspiration lumen extends through the elongate shaft. One or more aspiration ports are fluidly coupled with the aspiration lumen. The one or more aspiration ports may be disposed proximally of the one or more injection ports.

In some cases, the catheter may include a collapsible filter that is secured to the elongate shaft near the one or more aspiration ports. In some cases, the catheter may include a delivery lumen that is adapted to accommodate an implantable medical device advanced within the delivery lumen. In some cases, the catheter includes a proximal hub. The proximal hub may include a first fitting that is fluidly coupled with the injection lumen and is adapted to be connected with a source of contrast media and a second fitting that is fluidly coupled with the aspiration lumen and is adapted to be connected with a source of vacuum.

A variety of different contrast media may be used. As an example, the contrast medial may be 1-N,3-N-Bis(2,3-dihydroxypropyl)-5-[N-(2,3-dihydroxypropyl)acetamido]-2,4,6-triiodobenzene-1,3-dicarboxamide, which is commonly known as iohexol. Iohexol is an Iodine-bearing molecule, and has the structure shown below:

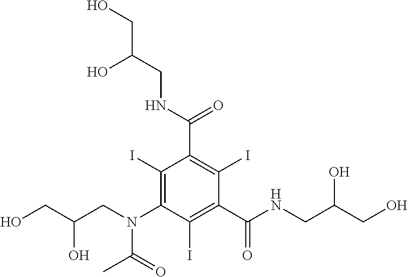 [SMILES: CC(=O)N(CC(O)CO)c1c(I)c(C(=O)NCC(O)CO)c(I)c(C(=O)NCC(O)CO)c1I]

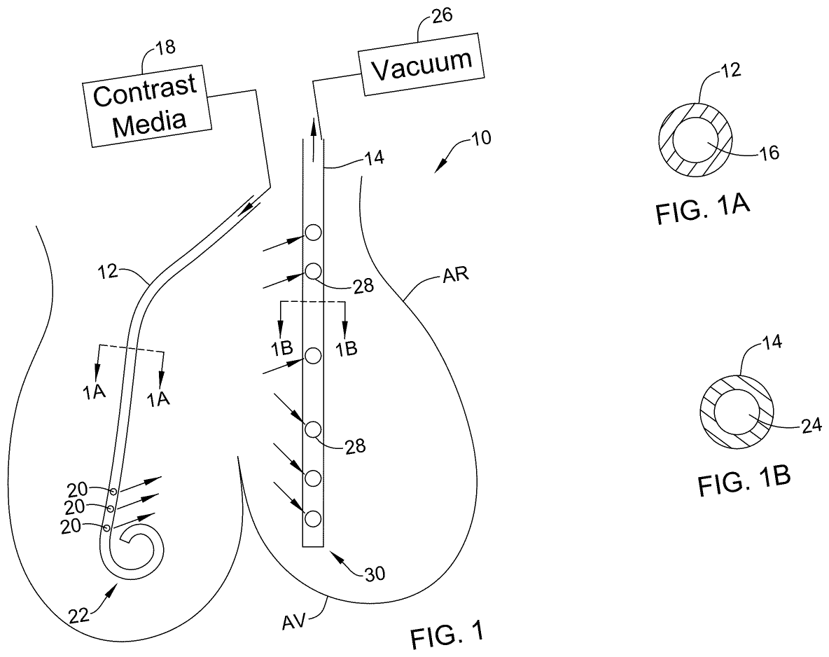

FIG. 1 is a schematic view of an illustrative system 10 for injecting and recapturing a contrast media such as iohexol. FIGS. 1A and 1B are cross-sectional views taken along lines 1A-1A and 1B-1B of FIG. 1. The illustrative system 10 is shown disposed within an aortic root AR, near an aortic valve AV. The anatomy is shown schematically. The illustrative system 10 includes an injection catheter 12 that is used to provide and inject a contrast media and an aspiration catheter 14 that is used to recapture at least some of the contrast media. As can be seen in FIG. 1A, the injection catheter 12 defines a lumen 16 that may be fluidly coupled with a source of contrast media 18. Returning to FIG. 1, it can be seen that the injection catheter 12 includes any number of (three are shown) injection ports 20 that are fluidly coupled with the lumen 16 such that any contrast media passing through the lumen 16 may exit through the one or more injection ports 20. In some cases, as shown, each of the injection ports 20 are located close together, and are near a distal region 22 of the injection catheter 12. In some cases, the injection ports 20 may be located farther apart. In some cases, the injection ports 20 may be located circumferentially about the injection catheter 12. The injection ports 20 may follow a helical path about the injection catheter 12, for example, or may be arranged in multiple straight lines of injection ports 20. A first line of injection ports 20 may be circumferentially spaced about 60 to 90 degrees from a second line of injection ports 20. In some cases, the distal region 22 of the injection catheter 12 includes a pigtail, as shown.

As can be seen in FIG. 1B, the aspiration catheter 14 defines a lumen 24 that may be fluidly coupled with a source of vacuum 26. Returning to FIG. 1, it can be seen that the aspiration catheter 14 has any number (six are shown) of aspiration ports 28 that are fluidly coupled with the lumen 24 such that the vacuum source 26, when fluidly coupled with the lumen 24, will cause fluids proximate the aspiration catheter 14 to be aspirated through the aspiration ports 28 and into the lumen 24. In some cases, the aspiration ports 28 may be located circumferentially about the aspiration catheter 14. The aspiration ports 28 may follow a helical path about the aspiration catheter 14, for example, or may be arranged in multiple straight lines of aspiration ports 28. A first line of aspiration ports 28 may be circumferentially spaced about 60 to 90 degrees from a second line of aspiration ports 28. In some cases, the aspiration ports 28 located relatively closer to the contrast injection site may be larger in diameter while the aspiration ports 28 that are located farther downstream may be smaller in diameter.

The fluids proximate the aspiration catheter 14 include blood and contrast media that is within the blood. Thus, at least some of the contrast media injected by the injection catheter 12 may be recaptured via the aspiration catheter 14. In some cases, there may be more aspiration ports 28 on the aspiration catheter 14 than there are injection ports 20 on the injection catheter 12. In some cases, the aspiration ports 28 may vary in size and/or location from what is shown. The aspiration catheter 14 includes a distal region 30. In some cases, as shown, the distal region 30 may be closed off. In some cases, the distal region 30 may include a pigtail (much like the pigtail shown as the distal region 22 of the injection catheter 12).

In some cases, there is a desire for a sufficient amount of contrast media to remain at a location proximate the injection catheter 12 and the aspiration catheter 14 for the contrast media to be effective in improving visibility during fluoroscopy. In some cases, fluoroscopy may occur while the contrast media is being injected by the injection catheter 12. The aspiration catheter 14 may start aspirating as soon as the injection catheter 12 starts injecting contrast media. In some cases, the aspirating catheter 14 may not start aspirating until a short delay after fluoroscopy has started, in order to improve visibility.

As shown, the injection catheter 12 and the aspiration catheter 14 are separate catheters. In some cases, the lumen 16 (shown as extending through the injection catheter 12) and the lumen 24 (shown as extending through the aspiration catheter 14) may both be disposed within a single catheter. In some cases, when using a single catheter, the injection ports 20 and the aspiration ports 28 may be axially spaced apart. For example, the injection ports 20 and the aspiration ports 28 may be located such that the aspiration ports 28 are downstream of the injection ports 20 when the catheter is deployed within the vasculature. This can allow a residence time for the injected contrast media at or near a desired location for fluoroscopically viewing the desired location before blood flow carries the injected contrast media to the aspiration ports 28.

In some cases, the contrast media (such as iohexol) may be combined with a magnetic material that allows the contrast media to be removed magnetically. As an example, the iohexol may be combined with ferromagnetic particles such as SPIONS (superparamagnetic iron oxide nanoparticles). Combining the iohexol with the SPIONS may include one or more of covalent bonding, hydrogen bonding and ionic bonding, for example. In some cases, SPIONs may have a diameter that ranges from about 85 nanometers to about 173 nanometers, although other sizes are contemplated. The SPIONS will be attracted to an electromagnetic or ferromagnetic membrane or surface. Electromagnetic refers to a surface that is not naturally magnetic, but functions as an electromagnet when a current is applied. Ferromagnetic refers to a surface that is naturally magnetic.

Individual iohexol molecules may be coupled to one or more of the SPIONS and the resulting iohexol-SPION complex will respond to a magnetic field. As an example, deploying a filter including an electromagnetic or ferromagnetic membrane within a blood stream will allow capture of at least some of the iohexol-SPION complexes that are in proximity to the filter. An electromagnetic or ferromagnetic membrane may be made from a work-hardened 316L austenitic stainless steel wire or other magnetic stainless steels having sufficient corrosion resistance and biocompatibility. The magnetic stainless steel may be used to manufacture a mesh-like membrane or “steel wool” like structure and may optionally be coated with an inert material. These materials may be adapted to be permanently magnetic, or may be electromagnetic if placed near an insulated coil having a current passing through the insulated coil. The membrane may be supported by supports that are made of a shape memory alloy such as nitinol.

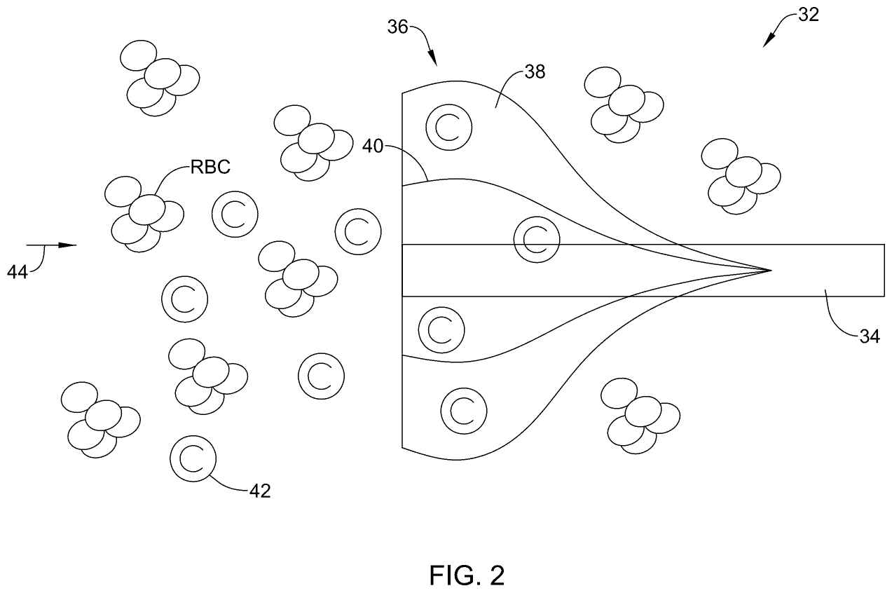

FIG. 2 is a schematic view of a system 32 that includes a shaft 34 and an electromagnetic filter 36 that is secured to the shaft 34. The shaft 34 may be part of a device that is adapted to inject contrast media, for example, as well as to possibly deliver an implantable medical device. In some cases, the electromagnetic filter 36 may be used in combination with the system 10 that is shown in FIG. 1. In this case, the shaft 34 may represent the aspiration catheter 14. In some cases, the electromagnetic filter 36 includes an electromagnetic membrane 38 that is secured to several supports 40. The supports 40 may be movable between a collapsed configuration that holds the electromagnetic membrane 38 against the shaft 34 for delivery and an expanded configuration (as shown) that allows the electromagnetic membrane 38 to be expanded. The supports 40 may be made from a shape memory material such as nitinol and thus may automatically expand when exposed to body temperature blood. In some cases, the supports 40 may instead be made of a material such as spring steel, and may be held in the collapsed configuration via sheath that extends over the electromagnetic membrane 38. Removing the sheath 38 allows the supports 40 to move into the expanded configuration.

In FIG. 2, red blood cells are labeled as RBC and the iohexol-SPION complexes are labeled as 42. Upstream of the electromagnetic filter 36, as indicated by an arrow 44 showing the direction of blood flow, iohexol-SPION complexes 42 intermingle with red blood cells RBC. As the blood enters the electromagnetic filter 36, the red blood cells RBC pass beyond the electromagnetic membrane 38 while the iohexol-SPION complexes 42 are drawn towards and are captured on the electromagnetic membrane 38. Downstream of the electromagnetic filter 36, the blood includes red blood cells RBC, but does not include any of the iohexol-SPION complexes 42 because the iohexol-SPION complexes 42 are captured on the electromagnetic membrane 38. In some cases, oxygenated blood within the aorta, for example, may actually be repelled by the electromagnetic membrane 38. While a single electromagnetic filter 36 is shown, in some cases multiple electromagnetic filters 36, each having differing pore sizes, may be deployed.

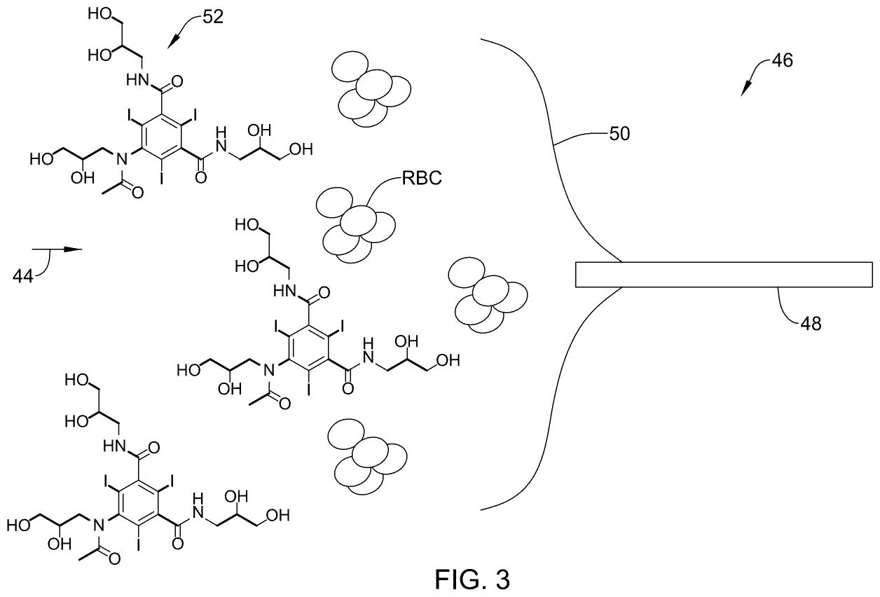

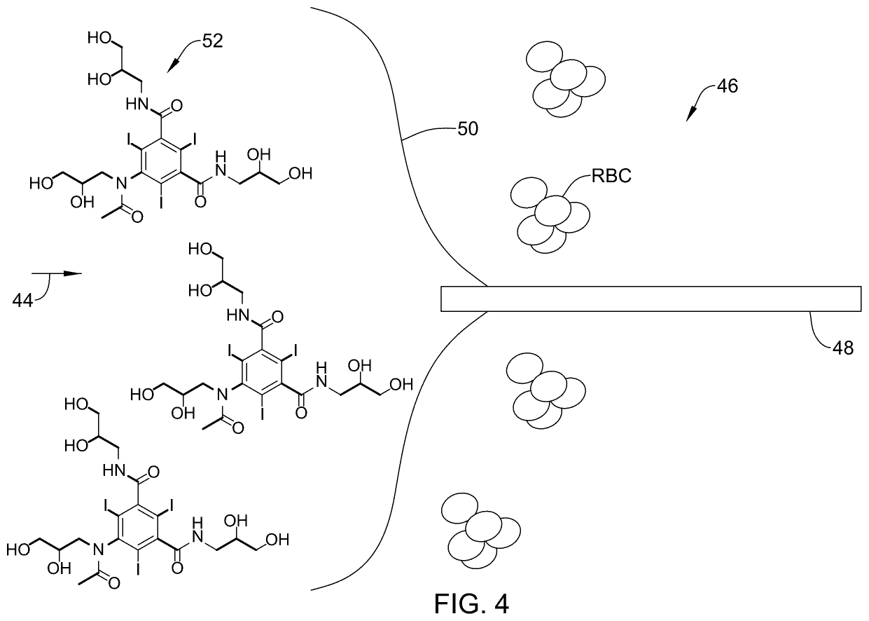

In some cases, the contrast media such as iohexol may be physically filtered out of the blood, in a process that is similar to the glomerular filtration that occurs naturally in the kidneys. FIG. 3 and FIG. 4 are schematic views of an illustrative physical filter before and after the contrast media has reached the physical filter. FIG. 3 is a schematic view of a system 46 that includes an elongate shaft 48 and an expandable physical filter 50 that is secured to the elongate shaft 48. In some cases, the physical filter 50 may be used in combination with the system 10 that is shown in FIG. 1. In this case, the elongate shaft 48 may represent the aspiration catheter 14.

While the expandable filter 50 is shown in an expanded configuration, it will be appreciated that the expandable filter 50 may be collapsed down onto the elongate shaft 48 for delivery, including advancement through the vasculature. The elongate shaft 48 may be part of a device that is adapted to inject contrast media, for example, as well as to possibly deliver an implantable medical device. Upstream of the expandable filter 50, as indicated by the arrow 44 showing the direction of blood flow, iohexol molecules 52 intermingle with red blood cells RBC. However, as the blood enters the expandable filter 50, the red blood cells RBC pass through pores that are formed in the expandable filter 50 while the iohexol molecules 52 are captured by the expandable filter 50. The expandable filter 50 includes pores that are dimensioned to allow the red blood cells RBCs to pass through, as well as pores that are dimensioned to allow other blood components such as white blood cells to pass through. As shown in FIG. 4, the red blood cells RBC have passed through the expandable filter 50 while the iohexol molecules 52 do not pass through, and thus are captured by the expandable filter 50. The expandable filter 50 may be made of a variety of different organic materials or inorganic materials that are commercially available. Examples include nylon, PET (polyethylene terephthalate), cellulose nitrate, polycarbonate, polysulfone, silicone carbide and aluminum, among others. The expandable filter 50 may be woven or laser-cut prior to being attached to collapsible supports via welding or adhesive.

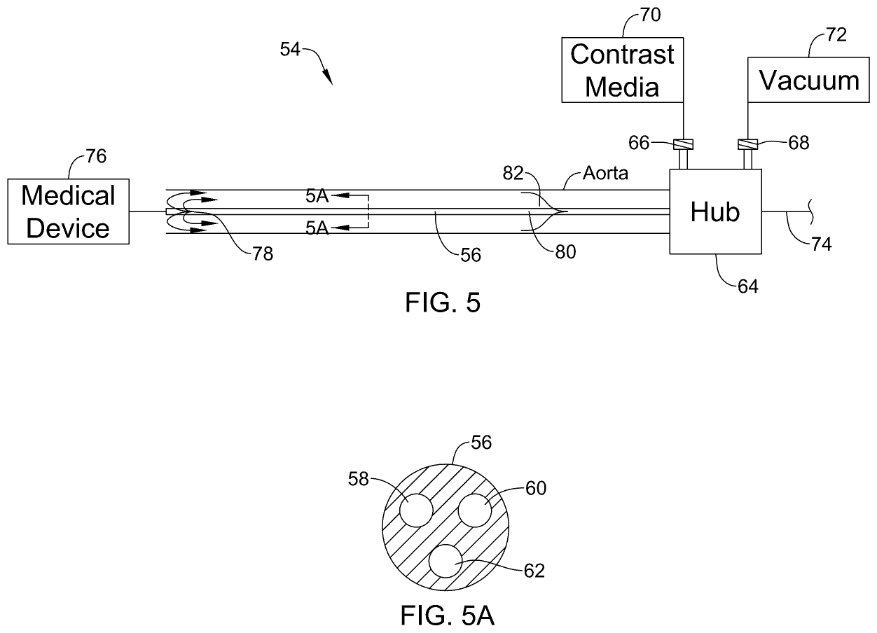

FIG. 5 is a schematic view of an illustrative system 54 that may be used to deliver contrast media and to then recapture at least some of the contrast media after a short period of time. The system 54 includes a catheter 56. As can be seen in FIG. 5A, which is a cross-sectional view along line 5A-5A of FIG. 5, the catheter 56 includes an injection lumen 58, an aspiration lumen 60 and a working lumen 62. It will be appreciated that this cross-sectional view merely shows an example catheter structure, as a variety of other catheter structures are also contemplated. In some cases, the catheter 56 may include a hub 64. The hub 64 may include a first fitting 66 and a second fitting 68. In some cases, the first fitting 66 and the second fitting 68 may be Luer fittings, but this is not required. The first fitting 66 may be fluidly coupled with the injection lumen 58, and may be adapted to be coupled with a source of contrast media 70. The second fitting 68 may be fluidly coupled with the aspiration lumen 60, and may be adapted to be coupled with a source of vacuum 72.

The working lumen 62 may be adapted to accommodate other devices. As an example, the working lumen 62 may be adapted to accommodate a delivery device 74 that includes a medical device 76 at a distal end of the delivery device 74. The medical device 76 may represent any of a variety of different implantable medical devices that may be implanted in a procedure that utilizes contrast media. As an example, the medical device 76 may represent a catheter-implantable replacement heart valve such as a replacement aortic valve.

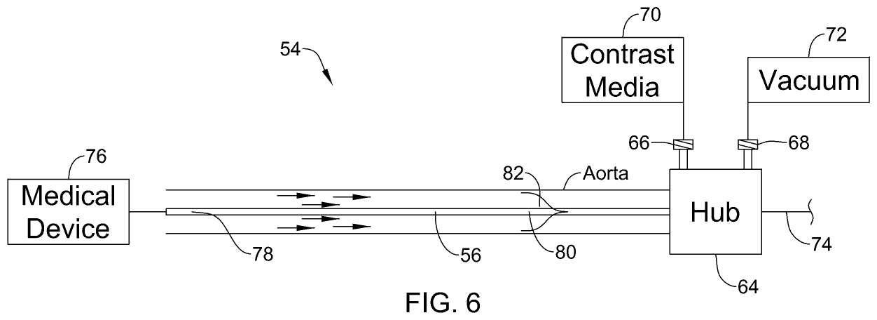

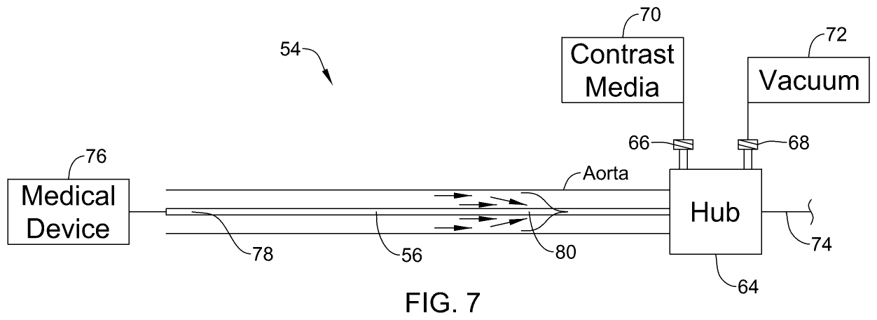

The catheter 56 includes one or more injection ports 78 and one or more aspiration ports 80. The one or more injection ports 78 extend through a wall of the catheter 56 and are fluidly coupled with the injection lumen 58. The one or more aspiration ports 80 extend through a wall of the catheter 56 and are fluidly coupled with the aspiration lumen 60. As shown in FIG. 5, contrast media is being injected through the first fitting 66, through the injection lumen 58 and out through the one or more injection ports 78. A physical or electromagnetic filter 82 (e.g., 36 or 50, as previously described) may be seen as being in an expanded configuration. In some cases, the filter 82 is positioned at or just proximal of the one or more aspiration ports 80. The vacuum source 72 has not yet been connected to the second fitting 68. FIG. 6 is a schematic view of the illustrative multi-lumen catheter of FIG. 5, shown after vacuum is applied and FIG. 7 is a schematic view of the illustrative multi-lumen catheter of FIG. 5, shown after the contrast media has been recaptured. In FIG. 6, blood flow, which is in a left-to-right direction as shown, has started to carry the injected contrast media towards the right. The vacuum source 72 is now connected to the second fitting 68. In FIG. 7, contrast media is no longer being injected through the injection ports 78. The contrast media has been aspirated through the one or more aspiration ports 80 into the aspiration lumen 60. In some cases, the catheter 56 may include a ribbed positioning tube that helps to prevent the aspiration lumen 60 from collapsing when vacuum is applied.

The materials that can be used for the various components of the devices described herein may include those commonly associated with medical devices. In some instances, the various components of the devices described herein may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, various components of the devices described herein of, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility may be imparted. For example, the various components of the devices described herein may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some instances, the various components of the devices described herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); antiproliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, antiplatelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A method for visualizing a location within a patient while reducing potential impact on the patient's anatomy, the method comprising:
injecting a contrast media into a location within the patient's vasculature;
viewing the location fluoroscopically; and
recapturing at least some of the injected contrast media.

2. The method of claim 1, wherein recapturing at least some of the injected contrast media occurs near the location.

3. The method of claim 1, wherein recapturing at least some of the injected contrast media occurs downstream of the location.

4. The method of claim 1, wherein the injecting step temporally overlaps with the recapturing step.

5. The method of claim 1, wherein the viewing step temporally overlaps with the recapturing step.

6. The method of claim 1, wherein the contrast media is injected into the location using a first lumen fluidly coupled with one or more injection ports.

7. The method of claim 6, wherein recapturing at least some of the injected contrast media near the location comprises using a second lumen fluidly coupled with one or more aspiration ports.

8. The method of claim 7, wherein the first lumen extends through a first catheter and the second lumen extends through a second catheter.

9. The method of claim 7, wherein the first lumen and the second lumen both extend through a single catheter, with the one or more injection ports disposed distally of the one or more aspiration ports.

10. The method of claim 1, wherein recapturing at least some of the injected contrast media comprises capturing the injected contrast media within a filter.

11. The method of claim 10, wherein the filter includes a collapsible frame.

12. The method of claim 10, wherein the contrast media comprises a contrast agent bound to magnetic particles, and the filter comprises an electromagnetic or a ferromagnetic surface.

13. The method of claim 12, wherein the contrast agent comprises iohexol.

14. The method of claim 12, wherein the magnetic particles comprise superparamagnetic iron oxide nanoparticles (SPIONs).

15. An assembly for visualizing a location within a patient while reducing potential impact on the patient's anatomy, the assembly comprising:
an injection catheter including:
an injection lumen extending therethrough;
one or more injection ports fluidly coupled with the injection lumen for injecting contrast media; and
an aspiration catheter including:
an aspiration lumen extending therethrough; and
one or more aspiration ports fluidly coupled with the aspiration lumen.

16. The assembly of claim 15, wherein:
the injection lumen is adapted to be fluidly coupled with a source of contrast media; and
the aspiration lumen is adapted to be fluidly coupled with a source of vacuum.

17. A catheter, comprising:
an elongate shaft;
an injection lumen extending through the elongate shaft;
one or more injection ports fluidly coupled with the injection lumen for injecting contrast media;
an aspiration lumen extending through the elongate shaft; and
one or more aspiration ports fluidly coupled with the aspiration lumen, the one or more aspiration ports disposed proximally of the one or more injection ports.

18. The catheter of claim 17, further comprising a collapsible filter secured to the elongate shaft near the one or more aspiration ports.

19. The catheter of claim 17, further comprising a delivery lumen adapted to accommodate an implantable medical device advanced within the delivery lumen.

20. The catheter of claim 17, further comprising a proximal hub, the proximal hub including:
a first fitting fluidly coupled with the injection lumen and adapted to be connected with a source of contrast media; and
a second fitting fluidly coupled with the aspiration lumen and adapted to be connected with a source of vacuum.
